(19) United States
(12) Patent Application Publication
(10) Pub. No.: US 2015/0251015 A1
(43) Pub. Date: Sep. 10, 2015

(54) BIOELECTROMAGNETIC CIRCUIT DEVICE

(71) Applicants: GENIE HOUSE CO., LTD., Hsinchu City (TW); G-SYN Biotechnology CO., LTD., Tainan City (TW)

(72) Inventor: Su-Chen Chang, Hsinchu City (TW)

(21) Appl. No.: 14/637,703

(22) Filed: Mar. 4, 2015

(30) Foreign Application Priority Data
Mar. 4, 2014 (TW) 103107171

(51) Int. Cl. A61N 2/00; A61N 2/02

(52) U.S. Cl. CPC A61N 2/004; A61N 2/02

(57) ABSTRACT

The present disclosure provides a bioelectromagnetic circuit device, used for emitting a bioelectromagnetic wave having a particular frequency, so as to improve biological blood circulation, including: an electromagnetic circuit module, for generating a bioelectromagnetic wave having a particular frequency of 800 MHz to 1 GHz; an electric energy storage module, electrically connected to the electromagnetic circuit module, and used for storing electric energy and providing the electric energy to drive the electromagnetic circuit module; an antenna, electrically connected to the electromagnetic circuit module, and used for emitting the bioelectromagnetic wave externally; and an insulator, used for fixing the antenna, the electric energy storage module and the electromagnetic circuit module.

### CROSS-REFERENCE TO RELATED APPLICATION

This application claims the benefit of Taiwan Patent Application No. 103107171, filed on Mar. 4, 2014, which is hereby incorporated by reference for all purposes as if fully set forth herein.

### BACKGROUND

1. Technical Field

The present invention relates to a bioelectromagnetic circuit device; and particularly to a bioelectromagnetic circuit device that can emit an electromagnetic wave having a particular frequency, and by emitting a bioelectromagnetic wave having a particular frequency to tune an electromagnetic field of a creature, the bioelectromagnetic circuit device improves blood circulation of the creature and improves health of the creature.

2. Related Art

An electromagnetic field is a general term for a combination of an electric field and a magnetic field; the magnetic field is produced as the electric field changes over time, and the electric field is produced as the magnetic field changes over time; moreover, in an organism, various electromagnetic properties also exist at all scales from as small as biochemical and electrochemical reactions to as big as nervous systems, for example, neural signals of the brain are transmitted in a form of electric waves having a particular frequency. For another example, the heart and the gastrointestinal tract control muscle contraction and peristalsis also by using electric waves having a particular frequency. For another example, proteins are most important components for forming various cells, enzymes, growth factors, tissues or organs of organisms, and may also generate different electromagnetic properties due to different amino acid structures. For another example, a bioelectric phenomenon caused by a potential difference inside and outside a cell membrane and the flow of ions inside or outside the cell also has an electromagnetic property. All the examples described above show that the electromagnetic field is closely related to biological activities.

People began to observe the correlation between electromagnetic waves and biological phenomena long ago. There are many products using electromagnetic fields to improve human health on the market at present, which mainly use magnets, germanium, titanium or crystal as major materials to make up bracelets, necklaces or accessories asserted to be capable of producing particular electromagnetic fields, infrared rays or negative ions, and the like, to promote blood circulation of creatures and help improve health of the creatures. However, action principles of these products are not clearly proved yet, and it is unclear the electromagnetic wave at which frequency band, among the broad frequency bands of electromagnetic waves output by the products, can act upon which biological phenomenon. Such a situation is similar to the situation in traditional medical treatment where herbal medicine is effective but it is unclear which one of the rich components contained in the herbal medicine is a functional component. In addition, such electromagnetic products also lack continuous energy sources, because none of the magnets, germanium, titanium and crystal can actively absorb external energy or powered by an external energy supply. As a result, power of the electromagnetic fields produced by the magnets, germanium, titanium and crystal is attenuated continuously over time; the magnets, germanium, titanium and crystal cannot continuously supply energy or stabilize the output power of the products, and will be ineffective over time, and therefore, have no significant effects on health.

In addition, the magnets, germanium, titanium and crystal are natural ores, and their electromagnetic properties are easily affected by changes in their purity or by different crystalline structures; as a result, the output electromagnetic frequency is unstable, making the efficacy unstable.

In the prior art, for example, the R.O.C Patent No. M437172 discloses a fitness plate, which is a microelectronic product, including an integrated circuit, a printed antenna and at least one resin layer. The printed antenna is connected to the integrated circuit, and the resin layer wraps the integrated circuit and the printed antenna, so as to protect the integrated circuit and the printed antenna. In this way, the fitness plate can generate a radio frequency to tune biological electric fields of human bodies, so as to improve blood circulation and enhance health of users.

However, in the prior art, the fitness plate only widely absorbs and then remaps external electromagnetic waves; on one hand, the emitted frequency is uncertain, and the prior art does not mention that the electromagnetic wave generated by the fitness plate is at a particular frequency, and therefore, the product does not have a stable output value; on the other hand, the prior art does not mention that the product can actively absorb external electromagnetic waves, convert the electromagnetic wave to electricity and store the electricity so that the power of the electromagnetic field produced will not increase or decrease with the unstable electric energy acquired.

### SUMMARY

An objective of the present invention is mainly to provide a bioelectromagnetic circuit device at an electromagnetic wave frequency band between 800 MHz and 1 GHz, which receives an external electromagnetic wave by means of an antenna, converts the external electromagnetic wave to electric energy and stores the electric energy in an electric energy storage module, then provides the electric energy by means of the electric energy storage module to drive an electromagnetic circuit module to generate a bioelectromagnetic wave having a particular frequency of 800 MHz to 1 GHz, and emits the bioelectromagnetic wave to an external organism through the antenna, so as to improve blood circulation of the organism, and enhance health of a creature. In addition, the present invention uses an insulator to fix and protect the electromagnetic circuit module, the electric energy storage module and the antenna, so that the bioelectromagnetic circuit device does not easily damage the electromagnetic circuit module, the electric energy storage module or the antenna due to vibration.

Further, the bioelectromagnetic circuit device of the present invention uses the principle of inductive coupling to convert the external electromagnetic wave received by the antenna to electric energy, and stores the electric energy in the electric energy storage module. When the bioelectromagnetic wave output by the bioelectromagnetic circuit device is attenuated, the electric energy stored in the electric energy storage module is provided, so as to drive the bioelectromagnetic circuit device to output a bioelectromagnetic wave, and in this way, electric energy can be supplemented constantly by receiving an external electromagnetic wave, so as to maintain a stable output power of the bioelectromagnetic circuit device.

Further, the present invention further includes a matching module, which uses the principle of impedance matching to match a receiving frequency of the antenna with a frequency of the external electromagnetic wave, that is, automatically adjusts input impedance of the bioelectromagnetic circuit device according to the frequency of the external electromagnetic wave, to optimize a power of the antenna after the antenna receives the external electromagnetic wave, so as to increase conversion efficiency of the electric energy.

The present invention provides a bioelectromagnetic circuit device, including: an electromagnetic circuit module, for generating a bioelectromagnetic wave having a particular frequency of 800 MHz to 1 GHz; an electric energy storage module, electrically connected to the electromagnetic circuit module, and used for storing an electric energy and providing the electric energy to drive the electromagnetic circuit module; an antenna, electrically connected to the electromagnetic circuit module, and used for emitting the bioelectromagnetic wave externally; and an insulator, used for fixing the antenna, the electric energy storage module and the electromagnetic circuit module.

As described above, in one embodiment, the antenna is further electrically connected to the electric energy storage module, and is used for receiving an external electromagnetic wave in the air, converting the external electromagnetic wave to the electric energy, and outputting the electric energy to the electric energy storage module. The bioelectromagnetic circuit device further includes a matching module which is electrically connected to the antenna and used for optimizing a power at which the antenna receives the external electromagnetic wave.

As described above, in one embodiment, the antenna is a printed antenna, a single-core antenna and/or a multi-core antenna.

As described above, in one embodiment, the antenna is of gold or copper.

As described above, in one embodiment, the insulator is of polyethylene and/or ethylene terephthalate.

As described above, in one embodiment, the particular frequency is between 850 MHz and 950 MHz.

The bioelectromagnetic circuit device of the present invention receives an external electromagnetic wave by means of an antenna, converts the external electromagnetic wave to electric energy and stores the electric energy in an electric energy storage module, then provides the electric energy by means of the electric energy storage module to drive an electromagnetic circuit module to generate a bioelectromagnetic wave having a particular frequency of 800 MHz to 1 GHz, and emits the bioelectromagnetic wave to an external organism through the antenna, so as to tune an electromagnetic field of a creature, improve blood circulation of the creature, and enhance health of the creature.

The electric energy storage module of the present invention stores the received electric energy. When the bioelectromagnetic wave output by the bioelectromagnetic circuit device is attenuated, the electric energy stored in the electric energy storage module is provided, so as to maintain a stable output power and enhance the stability of the output power of the bioelectromagnetic circuit device.

The present invention further includes a matching module, which matches a receiving frequency of the antenna with a frequency of the external electromagnetic wave in the air, to optimize the power at which the antenna receives the electromagnetic wave in the air.

### BRIEF DESCRIPTION OF DRAWINGS

FIG. 1 is a three-dimensional diagram of a bioelectromagnetic circuit device according to the present invention;

FIG. 2 is a block diagram of a bioelectromagnetic circuit device according to the present invention; and

FIG. 3 is a block diagram of a matching module of a bioelectromagnetic circuit device according to the present invention.

### DETAILED DESCRIPTION

The objectives, structural features and functions of the present invention will be further understood from the description provided below with reference to the related embodiments and accompanying drawings.

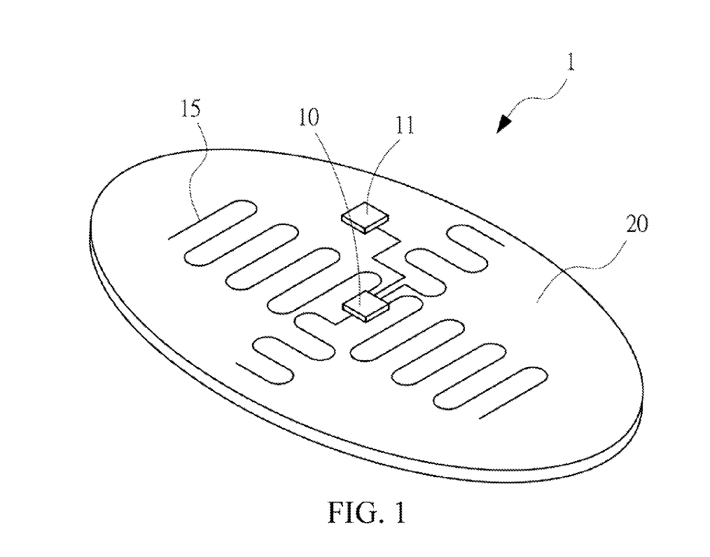

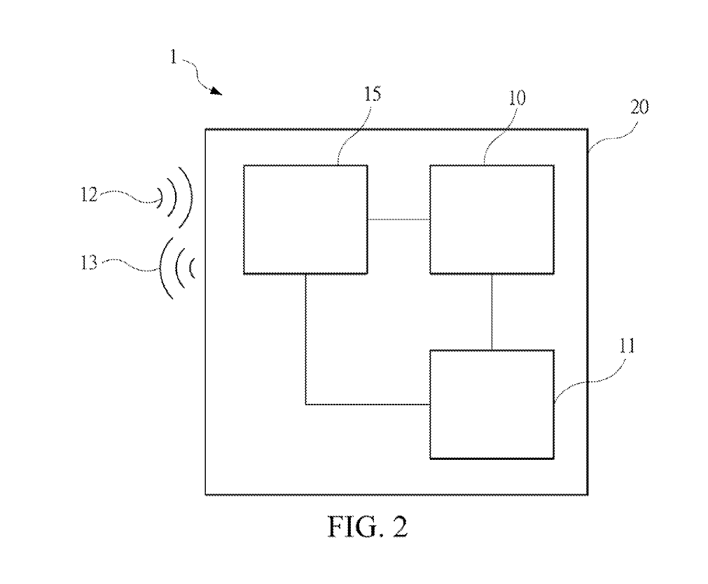

First, referring to FIG. 1 and FIG. 2, FIG. 1 is a three-dimensional diagram of a bioelectromagnetic circuit device according to the present invention; and FIG. 2 is a block diagram of a bioelectromagnetic circuit device according to the present invention. The present invention provides a bioelectromagnetic circuit device 1, including: an electromagnetic circuit module 10, for generating a bioelectromagnetic wave having a particular frequency of 800 MHz to 1 GHz; an electric energy storage module 11, electrically connected to the electromagnetic circuit module 10, and used for storing electric energy and providing the electric energy to drive the electromagnetic circuit module 10; an antenna 15, electrically connected to the electromagnetic circuit module 10, and used for receiving the bioelectromagnetic wave generated by the electromagnetic circuit module 10, and emitting the bioelectromagnetic wave 13 externally; and an insulator 20, used for fixing the antenna 15, the electric energy storage module 11 and the electromagnetic circuit module 10, so that the bioelectromagnetic circuit device 1 is not easily damaged due to vibration.

As described above, in one embodiment, the antenna 15 is further electrically connected to the electric energy storage module 11. The antenna 15 is used for receiving an external electromagnetic wave 12 in the air, converting the external electromagnetic wave 12 to the electric energy, and outputting the electric energy to the electric energy storage module 11, so as to store the electric energy, and when the power of the bioelectromagnetic wave output by the electromagnetic circuit module 10 is attenuated, the electric energy storage module 11 provides the stored electric energy to drive the electromagnetic circuit module 10, so as to maintain stable output of the bioelectromagnetic wave 13, for use by users.

As described above, in one embodiment, the antenna 15 is a printed antenna, a single-core antenna and/or a multi-core antenna.

As described above, in one embodiment, the antenna 15 is of gold or copper, but the present invention is not limited thereto, and the antenna 15 is of any conductive material.

As described above, in one embodiment, the insulator 20 is of polyethylene and/or ethylene terephthalate, but the present invention is not limited thereto, and the insulator 20 is of any polymeric material, the hardness of which is sufficient for fixing the antenna 15, the electric energy storage module 11 and the electromagnetic circuit module 10.

As described above, in one embodiment, the particular frequency of the bioelectromagnetic wave 13 is between 800 MHz and 1 GHz, and the optimal particular frequency of the present invention is between 850 MHz and 950 MHz, but the present invention is not limited thereto, and according to physical conditions of a user, the electromagnetic circuit module 10 can adjust the particular frequency of the bioelectromagnetic wave 13 to a frequency value beneficial to the health of the user.

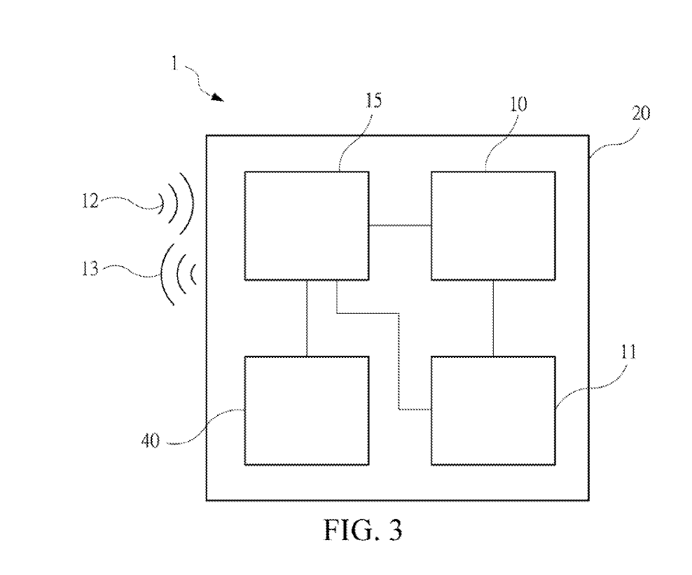

As described above, in one embodiment, the bioelectromagnetic circuit device 1 further includes a matching module 40. Referring to FIG. 3, FIG. 3 is a block diagram of a matching module of a bioelectromagnetic circuit device according to the present invention. The matching module 40 is electrically connected to the antenna 15, and the matching module 40 matches a receiving frequency of the antenna 15 with a frequency of the external electromagnetic wave 12 in the air according to the principle of impedance matching, that is, the matching module 40 automatically adjusts input impedance of the bioelectromagnetic circuit device 1 mainly based on the frequency of the external electromagnetic wave 12 in the air, matches the receiving frequency of the antenna 15 with the frequency of the external electromagnetic wave 12 in the air, and optimizes a power for receiving the external electromagnetic wave 12.

As described above, in one embodiment, the insulator 20 is further used for fixing the matching module 40.

Blood circulation is a basis for maintaining biological phenomena of an organism; slow blood circulation may cause cell hypoxic ischemia, which, in a less severe case, degrades performance of cells, and in a severe case, leads to cell lesion or necrosis. An experiment is carried out on two testees respectively by using the bioelectromagnetic circuit device of the present invention, a test is further carried out with a test instrument, namely, a non-intrusive arteriosclerosis screening device Colin VP2000, to measure changes in the arterial blood flow velocity before and after the testees use a chip, and testing results are as follows:

TABLE 1

| | Arterial blood flow velocity | Arterial blood flow velocity |
|---|---|---|
| | before the experiment | after the experiment |
| Testee | Right artery 1244 cm/s | Right artery 1361 cm/s, |
| 1 | Left artery 1253 cm/s | Left artery 1331 cm/s |
| | Blood pressure values of the | Blood pressure values of the |
| | right atrium: the systolic | right atrium: the systolic |
| | pressure is 134 and the | pressure is 125 and the |
| | diastolic pressure is 89; blood | diastolic pressure is 87; blood |
| | pressure values of the left : | pressure values of the left |
| | atrium the systolic pressure | atrium: the systolic pressure |
| | is 129 and the | is 125 and the |
| | diastolic pressure is 88 | diastolic pressure is 84 |
| Testee | Right artery 1252 cm/s | Right artery 1291 cm/s |
| 2 | Left artery 1271 cm/s | Left artery 1295 cm/s |

It can be known from Table 1 that Testee 1 is a hypertensive patient; upon comparison before and after the use of the bioelectromagnetic circuit device, before the bioelectromagnetic circuit device is used, the right arterial blood flow velocity of Testee 1 is 1244 cm/s and the left arterial blood flow velocity is 1253 cm/s; then the bioelectromagnetic circuit device is placed on the chest and two hands, and after eight minutes, the right arterial blood flow velocity is increased to 1361 cm/s and the left arterial blood flow velocity is increased to 1331 cm/s. Testee 2 is a normal, healthy subject, the testing result is shown in Table 1, which is not described herein again. It can be learned from Table 1 that, after the use of the bioelectromagnetic circuit device, the right and left arterial blood flow velocities are both improved significantly. As described above, within a short period of 8 minutes, the blood supply quantity for body tissue cells is increased by more than 5% on average, and a change of 9.5% is achieved for the patient, which has reached the degree of a significant medical change.

In addition, changes in the blood pressure before and after the use of the bioelectromagnetic circuit device are measured; before the use of the bioelectromagnetic circuit device, in terms of the blood pressure values of the right atrium of Testee 1, the systolic pressure is 134 and the diastolic pressure is 89, and in terms of the blood pressure values of the left atrium, the systolic pressure is 129 and the diastolic pressure is 88; after the use of the bioelectromagnetic circuit device, in terms of the blood pressure values of the right atrium, the systolic pressure is 125 and the diastolic pressure is 87, and in terms of the blood pressure values of the left atrium, the systolic pressure is 125 and the diastolic pressure is 84; after the measurement, it is found that the blood pressure does not increase evidently.

It can be learned from the above that an increase in the right and left arterial blood flow velocities does not rely on an increase in the burden of the heart, which also indicates that the bioelectromagnetic circuit device of the present invention can improve blood circulation throughout our body.

During actual use, the bioelectromagnetic circuit device of the present invention is attached to or carried on human bodies; bioelectromagnetic waves generated by the bioelectromagnetic circuit device interact with and act on an electromagnetic field of a creature, and particularly, the bioelectromagnetic circuit device is placed on a key energy field of the creature, for example, hands or feet, and at this time, the bioelectromagnetic circuit device will actively coordinate biological energy frequencies generated by the creature, to regulate blood circulation of the creature.

The bioelectromagnetic wave output by the bioelectromagnetic circuit device of the present invention is within a particular frequency between 800 MHz to 1 GHz and is further between 850 MHz to 950 MHz, and the bioelectromagnetic wave is output with a stable frequency value, therefore avoiding unstable efficacy, and blood circulation of the creature can be tuned effectively; in addition, the bioelectromagnetic circuit device further includes an electric energy storage module, which can store electric energy, and when the power of the bioelectromagnetic wave output by the bioelectromagnetic circuit device declines, the electric energy storage module provides the electric energy to stabilize the output power; finally, the bioelectromagnetic circuit device further includes a matching module which matches a receiving frequency of an antenna with a frequency of an external electromagnetic wave in the air, so as to optimize a power at which the antenna receives the electromagnetic wave in the air, and enhance conversion efficiency of the electric energy.

The above merely describes implementation manners or embodiments of technical means used in the present invention to solve the problems, which are not intended to limit the scope of implementation of the present invention patent. Equivalent variations and modifications conforming to the meaning of the patent application scope of the present invention or made according to the patent scope of the present invention shall fall within the patent scope of the present invention.

1. A bioelectromagnetic circuit device, comprising:
an electromagnetic circuit module, for generating a bioelectromagnetic wave having a particular frequency of 800 MHz to 1 GHz;
an electric energy storage module, electrically connected to the electromagnetic circuit module, and used for storing electric energy and providing the electric energy to drive the electromagnetic circuit module;
an antenna, electrically connected to the electromagnetic circuit module, and used for emitting the bioelectromagnetic wave externally; and
an insulator, used for fixing the antenna, the electric energy storage module and the electromagnetic circuit module.

2. The bioelectromagnetic circuit device according to claim 1, wherein the antenna is further electrically connected to the electric energy storage module, and is used for receiving an external electromagnetic wave in the air, converting the external electromagnetic wave to the electric energy, and outputting the electric energy to the electric energy storage module.

3. The bioelectromagnetic circuit device according to claim 1, wherein the antenna is a printed antenna, a single-core antenna and/or a multi-core antenna.

4. The bioelectromagnetic circuit device according to claim 1, wherein the antenna is of gold or copper.

5. The bioelectromagnetic circuit device according to claim 1, wherein the insulator is of polyethylene and/or ethylene terephthalate.

6. The bioelectromagnetic circuit device according to claim 1, wherein the particular frequency is further between 850 MHz and 950 MHz.

7. The bioelectromagnetic circuit device according to claim 2, further comprising a matching module, electrically connected to the antenna, and used for optimizing a power at which the antenna receives the external electromagnetic wave.
